(19) United States
(12) Patent Application Publication
(10) Pub. No.: US 2025/0288406 A1
(43) Pub. Date: Sep. 18, 2025

(54) NOVEL ENHANCED PROCESSES AND METHODS FOR CLOT/THROMBUS DIVERSION WITHIN THE CAROTID ARTERIES

(71) Applicant: Boris Leonardo Pabon Guerrero, Medellin (CO)

(72) Inventor: Boris Leonardo Pabon Guerrero, Medellin (CO)

(21) Appl. No.: 19/078,747

(22) Filed: Mar. 13, 2025

Related U.S. Application Data
(60) Provisional application No. 63/564,634, filed on Mar. 13, 2024.

(51) Int. Cl. A61F 2/01; A61F 2/00; A61F 2/06; A61F 2/90; A61F 2/954

(52) U.S. Cl. CPC A61F 2/011; A61F 2/954; A61F 2002/0081; A61F 2002/016; A61F 2002/068; A61F 2/90

(57) ABSTRACT

An intravascular device reduces stroke risk attendant passage of a clot originating in a common carotid artery. The device includes a stent adapted for arterial placement intravascularly at a junction at which the common carotid artery branches into an interior carotid artery and an external carotid artery. The stent includes a generally tubular configuration having a length and an outer encircling wall, thereby defining a first opening at a proximal end and a second opening at a distal end. A passage defined by said tubular configuration communicatively interconnects the first and second openings to provide a flow path for blood flowing into the first opening from the common carotid artery when the stent is placed at the junction. The stent wall includes a porosity structured to allow a flow of blood filtered by the wall passing across the wall while inhibiting passage of emboli larger than a predetermined size.

### CROSS REFERENCE TO RELATED APPLICATIONS

This application claims the benefit of U.S. Provisional Application No. 63/564,634 filed Mar. 13, 2024 entitled NOVEL ENHANCED PROCESSES AND METHODS FOR CLOT/THROMBUS DIVERSION WITHIN THE CAROTID ARTERIES.

### FIELD OF THE INVENTION

The invention relates to compositions, devices, and methods of minimally emplaced devices, especially as it relates to diversion of a clot/thrombus or thrombi within subject blood vessels, particularly the carotid arteries.

### BACKGROUND OF THE INVENTION

The background description includes information that may be useful in an understanding of the present invention, and is not intended to serve as an admission that any of the information provided herein is other than prior art or merely relevant as a backdrop to the presently claimed invention, or that any publication specifically or implicitly referenced is other than prior art.

During thrombectomy with antegrade blood flow, an engaged emboli generally in a form of a clot/thrombi, for example having been liberated from arterial plaque, can become dislodged particularly in a location of any stent/mechanical device. Blood flow can often have a water-hammer effect on the emboli, which can break pieces of the emboli off or entirely dislodge the emboli from the region of the capturing device. This occurrence becomes critical at the carotid ICA/ECA juncture in terms of travel or flow of the liberated clot/thrombi, since in absence of intervening diversion, the emboli could normally flow into the ICA, rather than into the ECA.

In most cases, the dislodged emboli or fragments thereof can either re-occlude the original vessel, i.e. the common carotid artery (CCA) prior to splitting into the ICA and ECA, or occlude a new vessel or territory beyond the junction of the branch. Hence, there has been a longstanding need to divert selective sizes of clots, particularly in the carotid branches beyond the carotid bulb located just past the ICA/ECA juncture. It is accepted science that the ECA is a much safer destination than the ICA for receiving any occurring larger dimensioned emboli, and thereby presenting the attendant risk of vessel occlusion, since the latter vessel branch leads to, and supplies blood, to the brain, whereas the ECA substantially supplies extracranial structures and therefore not posing a stroke risk.

Thus, there is a need for improved devices and methods available in the course of performing carotid stenting, which includes thrombectomy devices, and especially neurovascular thrombectomy devices, which meaningfully reduces incidence of stroke as a consequence of thrombectomy.

### SUMMARY OF THE INVENTION

The present inventive subject matter is drawn to compositions, devices, and methods of use to address the disease states impacting the brain, namely ECA travel for any larger clot/thrombi/emboli rather than conduction of same via the ICA.

Briefly stated, various embodiments of the invention operate to mechanically separate larger sized emboli from smaller emboli, directing passage of blood comprising larger emboli present, only into the ECA, while allowing only a filtered portion of supplied blood from the carotid artery having only any smaller clots that may be present, into the ICA.

An advantageous embodiment of the invention provides a stent which, when properly placed in the artery, bridges the CCA and the ECA so as to direct unfiltered blood received from the CCA into an opening of the stent at a proximal end and out through another stent opening at a distal end of the stent in communication with the ECA.

The stent according to embodiment of the invention diverts flow of unfiltered blood received directly through the stent from the CCA into the ECA branch, and includes a porous wall structure which filters out any emboli larger than a predetermined size from the blood entering the stent through the opening of the stent at the proximal end, across the stent wall in communication with the carotid bulb at the entry of the ICA.

Various objects, features, aspects and advantages of the inventive subject matter will become more apparent from the following detailed description of advantageous embodiments, along with the accompanying drawing in which like numerals represent like components.

### BRIEF DESCRIPTION OF THE DRAWING

FIG. 1 is a detailed cross-sectional view of a carotid arterial junction including an exemplary ICA clot/thrombus diverter stenting device positionally installed intravascularly according to the invention.

### DETAILED DESCRIPTION OF THE INVENTION

A clot/thrombus diverting stenting device with improved implantation characteristics is disclosed, especially with improved embolus diversion into the ECA not the ICA. The present stent can be prepared by inventive modification of existing systems as claimed below and shown the FIGURE. Expressly incorporated herein by reference are U.S. Pat. Nos. 10,398,777; 9,808,359; 8,088,140; 8,518,545; 8,585,713; 8,197,543 and 8,070,791 and all publications and references therein, as if fully set forth herein.

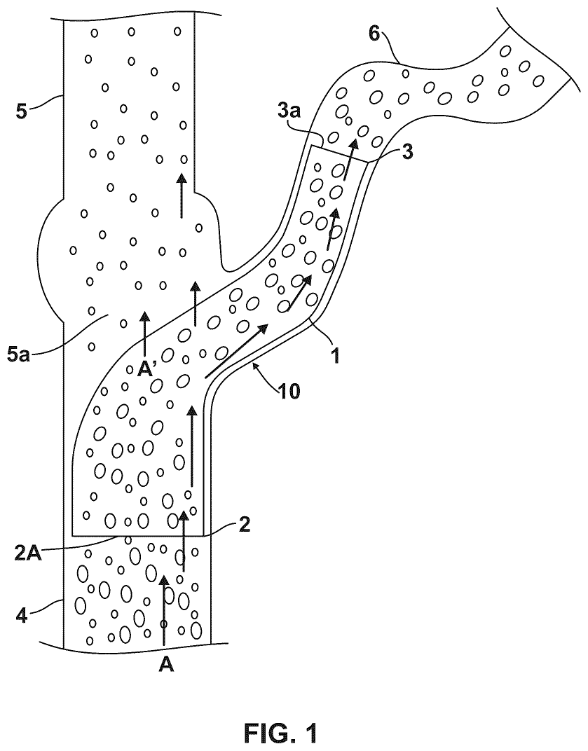

Referring to FIG. 1, a detailed cross-sectional view of a carotid arterial junction is depicted, and which shows a common carotid artery (CCA) 4 which splits into an internal carotid artery (ICA) 5 which includes a carotid bulb 5a adjacent to an entry of an external carotid artery (ECA) 6. As already mentioned, stroke risk is generally confined to adverse events occurring in ICA 5, since this artery branch serves to supply blood intracranially to the brain, and therefore embodiment of the invention intentionally diverts any larger emboli into the safer ECA 6, as will be described below herein.

A clot/thombosis diverter stent (referred to hereinafter simply as “stent”) is generally identified at 10. Stent 10 comprises a flexible, generally tube shaped configuration including a porous wall structure 1 extending between a first opening 2a at a proximal end 2 of stent 10 and a second opening 3a at a distal end 3 of stent 10. Wall structure 1 is configured to be porous to a degree inhibiting travel of larger emboli thereacross, while allowing filtered blood possibly having only smaller emboli to enter ICA 5.

Advantageously, a degree of flexibility of stent 10 will be sufficient to navigate and vascular tortuosity encountered in the intravascular placement thereof.

While stent 10 is optionally of a braided construction, it will be understood that any suitable construction providing the desired functional aspects described herein may be employed without departure from the invention.

Advantageously, although not necessarily, stent 10 is compatible with 0.021 or 0.027 inch microcatheters for the intravascular placement thereof. Filtering across wall structure 1 will advantageously allow passage of emboli having a size no larger than about 200-300 microns. Advantageously, stent 10 and a delivery system thereof allows repositioning as desired.

While a self-expanding design may be advantageously used for stent 10 configure to suit varying carotid artery internal diameters, any suitable type stent can be used within the context herein, including for example a stent design that is compatible with a balloon catheter suitably designed to achieve expansion of stent 10 into varying diameter portions of carotid artery internal geometry present in the intravascular placement position.

A direction and location of carotid artery blood flow is shown by two series of arrows in FIG. 1. Arrows A describe a flow from CCA 4 directed/diverted into ECA 6, and arrows A′ show filtered blood flow passing across porous wall structure 1 of stent 10 into ICA 5.

As depicted, blood flow A from CCA 4 flows into first opening 2a at proximal end 2 of stent 10, and flows out, unfiltered from second opening 3a at distal end 3 of stent 10, into ECA 6. Filtered blood flow A′ is directed into ICA 5 after being filtered by transport across porous wall structure 1.

It is noted that while stent 10 will find particular advantage in addressing a clot/trombi dislodged during a thrombectomy, for example emboli having been liberated from arterial plaque as discussed in the Background section herein, in which case placement of stent 10 is of limited duration, it is contemplated that stent placement may be of extended duration, and used to continuously filter entry of any potentially dangerous emboli produced for whatever reason, and exceeding predefined safe size limits, from entry into ICA 5. Such more permanent approach to use of stent 10 does not depart from the invention, and is deemed to be within the intended scope thereof.

A method of marketing a stent implemented by a vender or provider of the stent to a potential buyer thereof including a user, a medical facility or a medical provider for the user, is also contemplated withing the scope of the invention, intended to assign infringement to a vendor or provider who engages in practice of the marketing method. Therefore, any vendor or provider who instructs the potential buyer to use the stent in a manner placing the stent at a junction at which a common carotid artery branches into an interior carotid artery and an external carotid artery with a first stent opening of the stent being aligned to receive the flow of the blood received from the common carotid artery and orienting a second opening inward of the start of the external carotid artery such that the blood received from the common carotid artery flows out of the second opening of the stent, while positioning said outer encircling wall to span a start of the interior carotid artery such that blood in said passage such that the blood is filtered by passage across said wall to inhibit passage of emboli larger than a predefined size creating filtered blood which enters the interior carotid artery, could be held accountable for infringement without having to prove such actual use by the potential recipient.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment”, and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patent ability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e. g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, ?ash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e. g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An intravascular device for reducing stroke risk attendant passage of a clot originating in a common carotid artery, comprising:
a stent adapted for arterial placement intravascularly at a junction at which the common carotid artery branches into an interior carotid artery and an external carotid artery;
said stent comprising a generally tubular configuration having a length and an outer encircling wall, said stent including a first opening at a proximal end and a second opening at a distal end, a passage defined by said tubular configuration communicatively interconnecting said first and second openings to provide a flow path for blood flowing into said first opening from the common carotid artery when said stent is placed at the junction; and
said wall having a porosity structured to allow a flow of blood filtered by said wall passing across said wall while inhibiting passage of emboli larger than a predetermined size.

2. An intravascular device according to claim 1, wherein said wall comprises a braided material.

3. An intravascular device according to claim 1, wherein said stent is compatible with a 0.021″ and 0.072″ catheter for delivery to the arterial placement at the junction.

4. An intravascular device according to claim 1, wherein said porosity is selected to inhibit passage of a thrombosis no larger than about 200-300 microns.

5. An intravascular device according to claim 1, wherein said stent is self expanding to suit varying vessel internal diameters.

6. An intravascular device according to claim 1, wherein said stent is flexible along said length to navigate a vascular tortuosity.

7. A method of reducing or substantially eliminating incidence of strokes due to carotid-originating emboli, comprising:
directing unfiltered blood from a flow of the blood received from a common carotid artery into an external carotid artery; and
filtering said flow of the blood prior to permitting entry thereof into an interior carotid artery in a manner effective to remove any emboli larger than a predetermined size.

8. A method according to claim 7, further comprising:
providing a stent comprised of a generally tubular construction, said stent having a length and an outer encircling wall, said stent including a first opening at a proximal end and a second opening at a distal end;
placing said stent at the junction, with said first opening aligned to receive the flow of the blood received from the common carotid artery, a passage defined by said tubular configuration communicatively interconnecting said first and second openings to provide a flow path for blood flowing into said first opening from the common carotid artery;
orienting said second opening inward of the start of the external carotid artery such that the blood received from the common carotid artery flows out of the second opening of the stent; and
positioning said outer encircling wall to span a start of the interior carotid artery such that blood in said passage such that the blood is filtered by passage across said wall to inhibit passage of emboli larger than a predefined size creating filtered blood which enters the interior carotid artery.

9. A method of marketing a stent implemented by a vender or provider of the stent to a potential recipient thereof including a user, a medical facility or a medical provider for the user, the marketing method comprising:
instructing the potential buyer to place the stent comprised of a generally tubular construction and having a length and an outer encircling wall, said stent including a first opening at a proximal end and a second opening at a distal end at a junction at which a common carotid artery branches into an interior carotid artery and an external carotid artery, with said first opening aligned to receive the flow of the blood received from a common carotid artery, a passage defined by said tubular configuration communicatively interconnecting said first and second openings to provide a flow path for blood flowing into said first opening from the common carotid artery; and
further instructing orienting said second opening inward of the start of the external carotid artery such that the blood received from the common carotid artery flows out of the second opening of the stent, while positioning said outer encircling wall to span a start of the interior carotid artery such that blood in said passage such that the blood is filtered by passage across said wall to inhibit passage of emboli larger than a predefined size creating filtered blood which enters the interior carotid artery.

10. A method of marketing according to claim 9, wherein said instructing includes providing a use recommendation on the stent itself, by verbal or illustrative communication, by way of instructional media, and/or packaging and/or advertising material thereof for the stent.
